(19) United States
(12) Patent Application Publication
(10) Pub. No.: US 2019/0117939 A1
(43) Pub. Date: Apr. 25, 2019

(54) A Cannula Protector

(71) Applicant: RESQDEVICES PTY LTD, Eveleigh, NSW (AU)

(72) Inventors: Roger Price, Eveleigh (AU); Robert Seaman, Eveleigh (AU); Michael Potas, Eveleigh (AU); Giovani Ciampa, Eveleigh (AU)

(21) Appl. No.: 16/094,732

(22) PCT Filed: Apr. 18, 2017
(86) PCT No.: PCT/AU2017/050344

§ 371 Date: Oct. 18, 2018

(30) Foreign Application Priority Data
Apr. 18, 2016 (AU) 2016901451

(51) Int. Cl. A61M 25/02; A61M 5/14

(52) U.S. Cl. CPC A61M 25/02; A61M 5/1415; A61M 2025/0206; A61M 2025/0246; A61M 2005/1416; A61M 2209/088; A61M 2025/0253

(57) ABSTRACT

A cannula protector for protection of a cannulation site on a body is described. The protector includes a shield for inhibiting access to the cannulation site; and a body mount operatively connected to the shield, the body mount configured to provide one or more relief zones so as to relieve the mounting force of the protector when mounted on the body. A protector for protection of a cannulation site on a body is also described, the protector including: a shield for inhibiting access to the cannulation site; wherein the shield is configured to conform to a contour of the body to inhibit access to the cannulation site. Further, a protector for protection of a cannulation site on a body is described, the protector including: a shield for inhibiting access to the cannulation site; and a body mount operatively connected to the shield, the body mount including a plurality of body engaging feet; wherein the body engaging feet are configured to move to conform to a contour of the body to inhibit access to the cannulation site.

### PRIORITY

This application is the National Stage of International Application No. PCT/AU2017/050344 filed Apr. 18, 2017 which claims priority to Australian Application No. AU 2016901451 filed Apr. 18, 2016, and is incorporated herein by reference in its entirety.

### TECHNICAL FIELD

The present technology relates generally to a cannula protector for the protection of a cannula mounted on a body at a cannulation site. Cannulation sites are sites on a human or animal body on which a cannula or other intravenous device (IVD) is disposed in a deployed position for the intravenous delivery of treatment.

### BACKGROUND

About 15 million IVDs are used in Australia each year, and 330 million in the United States of America. Known related methods and devices for holding IVDs in place are inadequate, being as they are, generally speaking, unsophisticated arrangements of tape and bandages, sometimes integrated with rigid splint members for support. These arrangements take time to deploy and their longevity is at best variable since the mounting depends on human factors such as the skill, experience of the carer or nurse, and their ability to focus on the task at hand.

The abovementioned factors and others are likely to be a contributor to high IVD failure rates, which are estimated to affect about 40% of hospital patients. High IVD failure rates cost the Australian health system approximately $1 bn and 2 million nursing hours each year. The problem is worldwide.

One way of compensating for the high failure rate includes more tightly wrapping the splint members together, but this method can lead to reduction in IVD fluid delivery.

Generally speaking, the problem areas include one or more of:
fluid leakage from the cannula tip migrating from the vein into the tissue from micro-movement of the cannula;
dislodgement of the cannula, accidental or deliberate;
phlebitis (reddening of the site) due to localised irritation; and/or
inability to effectively monitor a cannula site.

Again, these issues are familiar to practitioners all around the world where IVDs are used; the costs increasing with population.

The present inventor seeks to provide an IVD protector/cannula retainer which ameliorates at least one of the above disadvantages, or at least provides an alternative cannula retainer or new retainer.

### SUMMARY

In a broad aspect there is provided a protector for protection of a cannulation site on a human or animal body portion, the protector including a shield, as well as one or more body mounts configured to relieve, in one or more selected relief zones, the protector mounting force on the body. In some embodiments the arrangement is such that the body mounts include one or more relief zones disposed adjacent a shield periphery in use to facilitate flow of fluid in the vessel of the body portion into which a cannula is inserted.

In another broad aspect there is provided a protector for protection of a cannulation site on a human or animal body, the protector being configured to conform to a contour of the body.

In yet another broad aspect there is provided a cannula protector for protection of a cannula on a cannulation site on a human or animal body, the cannula protector inhibiting patient access to the cannulation site but facilitating fluid access through cannulated veins into, out of and/or around the cannulation site.

In some embodiments the arrangement is such that the protector body mounts comprising one or more relief zones are disposed adjacent a protector periphery to facilitate flow of fluid in the vessel of the body into which a cannula is inserted.

In accordance with an aspect of the present technology there is provided a cannula protector for protection of a cannulation site on a body, the protector including:
a shield for inhibiting access to the cannulation site; and
a body mount operatively connected to the shield, the body mount configured to provide one or more relief zones so as to relieve the mounting force of the protector when mounted on the body.

The arrangement may be such that the body mount is configured as a plurality of body engaging mount elements or portions which are disposed adjacent or around a periphery of the shield, and at least two of the body engaging support elements or portions are spaced apart from one another to provide at least one of the relief zones therebetween. The arrangement may be considered to be mounting elements arranged in a broken shield perimeter. The breaks between the perimeter mounting elements may be disposed on a hinge line such that when in use they relieve pressure on a vein or artery under or adjacent the shield.

In accordance with another aspect of the present technology there is provided a protector for protection of a cannulation site on a body, the protector including:
a shield for inhibiting access to the cannulation site;
wherein the shield is configured to conform to a contour of the body to inhibit access to the cannulation site.

In one embodiment the shield includes a lid panel in the form of a plurality of lid elements overlapping one another and configured to move in a sliding relationship relative to one another to maintain a cover over the cannulation site when the shield is moved from a flat position to a conformal position.

In accordance with another aspect of the present technology there is provided a protector for protection of a cannulation site on a body, the protector including:
a shield for inhibiting access to the cannulation site; and
a body mount operatively connected to the shield, the body mount including a plurality of body engaging feet;
wherein the body engaging feet are configured to move to conform to a contour of the body to inhibit access to the cannulation site.

In one embodiment there is provided a body-engaging body mount connected to the shield, the body mount with the shield being configured to conform to a contour of the body.

### Body Mount

### Supports

In an embodiment the arrangement is such that the body mount includes a plurality of supports for supporting the shield in a selected position on the body. In one embodiment the supports include feet for distributing loads of the shield on the body. In one embodiment the feet are spaced apart from one another to provide stability to the shield and provide relief zones between each foot. In one embodiment, where there are provided shield vertices, the feet are spaced proximal opposed vertices of the shield for facilitating greater stability. In one embodiment the feet are elongate in that they include plates or plate-shaped foot elements for more widely distributing the loads of the shield on the body over a greater surface area of the body.

In one embodiment the feet include a fastener mount for mounting a fastener. In one embodiment the fastener mount is a webbing receiver for receiving a webbing element or webbing strap. The webbing receiver could be in some embodiments, an aperture, or some other form of guide including posts or channels, or other fastener mount that could be useful for holding the feet against a body. The fastener mount could be in some embodiments, an adhesive receiving area, or a tape hold-down area, or elastic band area, or other clamp for holding to a body to protect the cannulation site.

In one embodiment the feet include a pliable, spongy or soft material for further distributing stresses from particular portions of the feet on the body and so as to expand to fill any gaps which may be opened by undulations in the body adjacent the cannulation site. The pliable material in one embodiment is a pillow or air cushion. This gap-filling function further inhibits access to the cannulation site, since patients find it difficult to get under the feet, between the skin and the feet, to get to the cannula and cannulation site. The pliable material may be integrated with or applied to the surface of the feet. The pliable material may be EVA foam or other suitable foam or rubber, silicone, and spread evenly on the surface of the feet or applied in pads. There equally may be other pads or bosses or other extension pieces or fingers which when spread in discrete places on the feet, may distribute loads on the body. The bosses or fingers may be formed from a deformable material, or formed sufficiently finely so as to be flexible (plastic fingers, even metallic springs if suitably finely formed, and, say, leaved).

The EVA foam may be thick so as to provide one kind of relief zone, to spread the load of the feet and shield across the body, so as to facilitate patency of any vein or artery or other channel under or adjacent the shield or feet or protector generally.

In one embodiment the feet may themselves be sprung or in other ways configured to conform to some contour of the body adjacent the cannulation site. In some of these embodiments the feet may be articulated, supported themselves on springs or sprung posts, or hinged in a suitable way such as by having an integrated or living hinge. In these embodiments the feet may have an outer portion, in which the fastener mount is disposed, and which is distal the shield, and which is movable between a flat position and a flexed position to improve the distribution of the load, and/or the relief on any particular vein or artery or other vessel adjacent the skin surface, when required by the contours of the body adjacent the cannulation site.

A liner on the spongy or soft or pillow material may be provided to inhibit and/or absorb sweat developing under the pillow elements while in use. The liner may be made of woven textile, such as for example, cotton or technical fabric, and may be affixed to the spongy/pillow/cushion/foam material by adhesive, or it may be in the form of a sock, or some other suitable arrangement.

### Chassis

In one embodiment the support includes a chassis or frame to facilitate the spatial placement of other elements, including the shield. In one embodiment the chassis is disposed on the support.

In one embodiment the chassis is a peripheral frame arrangement having one or more frame members.

In some embodiments the chassis is configured to space the feet apart from one another around the chassis to facilitate stability of the shield.

In one embodiment the chassis includes chassis walls to inhibit access to the cannulation site. In one embodiment the chassis walls extend upwardly from the support. In some embodiments the chassis walls include spaced-apart side chassis walls and spaced-apart end chassis walls, operatively connected together. In one embodiment an opening at a rim is provided, the rim disposed at a top of the walls such that the IVD, or the cannulation site generally, can in use be accessed therethrough.

In some embodiments the chassis is configured to facilitate the shield and/or feet conforming to the body. In those embodiments at least some portions of the chassis, or the whole chassis, is articulated to facilitate the chassis moving between a straight position and a flexed position in which the chassis may conform to some convex surface such as a forearm. In those embodiments the chassis may be flexible in some way such that some chassis elements can be positioned in some selected flexed angular position relative to other chassis elements, or, some chassis portions can be positioned in some flexed angular position relative to other chassis portions. In one embodiment the chassis includes a chassis hinge to facilitate articulation. In one embodiment the chassis is generally rectangular with the side walls longer than the end walls, and a longitudinal axis extending between the end walls. The hinge may be in the form of a pair of pivots set into opposed end walls of the chassis so that a hinge line is extending therebetween, coaxial with the longitudinal axis, to facilitate articulation about that axis.

In one embodiment the chassis walls include one or more inlets for facilitating inlet of an IV line. In some embodiments there are four inlets provided, disposed in opposed vertices of the chassis. In some embodiments the inlets include buttresses to strengthen the chassis walls and to reduce snags for the IV line.

The inlets may be sized to be slightly wider than the IV line, so that a bung connecting a short cannula line to the IV line will be inhibited from being pulled through the chassis. The inlets may be set into the shield in addition to or alternative to the inlets on the chassis walls, which will provide the same retaining function.

It can be seen that the relief zones in one alternative are gaps between the feet, while in another alternative, or in addition to, can be soft, resilient, pillow, hollow cushion or foam or sponge elements to provide relief and relatively free flow of fluid in blood vessels into, out of, and/or around the feet, shield and chassis.

### Chassis-and-Support Interaction: Relief Zone

In one embodiment one foot is mounted at either end of a spaced-apart chassis side wall to facilitate stability of shield mounting. In some embodiments the foot extends along the longer side wall to increase the load over a larger area.

In some embodiments there is provided a foot extension pad between the two feet, which extends along an underside of each spaced-apart side wall to further inhibit access under the chassis wall to the cannulation site. The foot extension pad includes soft EVA foam as described above, but it may be a pillow or airbag or hollow air cushion.

In some embodiments the feet extend beyond the spaced-apart side chassis walls so as to inhibit access to the cannulation site and further spread the support load.

In one embodiment the spaced-apart chassis end walls are arranged such that there is a cut away in the chassis end walls, or the base edge extends upwards from the feet to provide pinch-free operation by making a little extra clearance for conforming, and thus one form of relief zone thereunder. That is, one embodiment of this relief zone is where there is an air gap disposed in use between the body and the body mount. The relief zone, when the protector is applied to the body, facilitates patency of a vein or artery or other fluid channel within the body, near the surface of the body, adjacent the cannula protector as well as facilitate the pivoting by providing extra clearance between at least a portion of the chassis and the body. This is useful to facilitate IVD fluid delivery to other parts of the body by maintaining vessel patency.

In one embodiment there is provided a relief zone in the form of a spongy area under one or more EVA foot pads.

In one embodiment the chassis hinge is disposed in each of the spaced-apart chassis end walls to form a chassis hinge line extending between the chassis end walls and parallel to the chassis side walls. In one embodiment the chassis hinge is arranged in the spaced-apart chassis end walls such that the chassis hinge is vertically spaced from the feet to inhibit pinching between feet when the chassis moves to a flexed position. The cutaway in the chassis end walls assists with pinch-free operation in use.

In one embodiment the feet are configured to extend along parallel to, and articulate along, coincident with, a foot hinge line which is parallel to the chassis hinge line so as to further facilitate conforming of the protector to the body.

The chassis end walls include a guide for the shield so that the shield may articulate in a way that is guided by the guide in the end walls. In one embodiment the guide includes an arcuate track along which a follower on the shield may follow.

Since the chassis end walls include the relief zone, the chassis end walls further include a flange disposed thereon to cover at least the higher portions of the relief zone to inhibit access to the cannulation site therethrough. The flange is in some embodiments integrated with the guide.

### Shield

In an embodiment the arrangement is such that the shield includes a closure which is configured to close the chassis rim opening, by adopting a closed position, to control access to the cannulation site or the IVD itself. In one embodiment the closure includes a lid panel. In one embodiment the lid panel also includes a skirt depending downwardly therefrom having opposed skirt walls configured to cooperate with the opposed chassis walls by being spaced outwardly therefrom and extending parallel thereto when in the closed position. The skirt further inhibits access to the cannulation site by at least partially covering the plurality of inlets, increasing its strength, and also by inhibiting a patient in prising off the lid panel from the chassis.

In an embodiment the closure is movable between the closed position and an access position. The closure may be movable between the closed position and the access position by being detachable, or slidable, or pivotable. In one embodiment the closure is pivotally attached to one chassis side wall to form a hinged closure to facilitate single-handed use of the protector.

In an embodiment the closure is pivotally attached to a spaced-apart side wall so as to facilitate ease of access to the cannulation site. The arrangement is such that the closure hinges on a line parallel to the chassis side wall line for simplicity of operation and for strength of hinge—since the hinges can be spaced further apart along the side wall than the end wall, the closure is more secure than if it were hinged on the end wall.

The shield provides visibility to the cannulation site when it is in the closed position by being transparent or translucent, or by having a window therein, or barred or mesh panels mounted on a peripheral lid frame, or other suitable arrangement for viewability. The shield may be made of ABS, Polycarbonate, or other suitable plastic.

The shield may include a plurality of pivoting lid panel elements, each one overlapping and sliding over the adjacent lid panel element. There may be one hinge, such that there are two lid panel elements, or there may be two or more hinges and an appropriate plurality of lid panel elements (=no. of hinges+1), so that there is a more conformal shield assembly.

The hinge may also be offset, so that the shield plates are not equal in size. In this embodiment, the chassis frame may also include a plurality of pivots and/or be in the form of a conformal frame element. Thus, the chassis frame may be a thinner malleable wire or ribbon or bar element made of metal, or nitinol, or other conformal material, coated in plastic, so that there may be a reduction in pressure points by virtue of the fact that conformation to the relevant body part is more complete.

The shield may also include the inlets as described.

### Expanding Closure

As mentioned, in an embodiment the closure is also configured to move with the chassis, the lid panel abutting the chassis rim, between a flat position and a flexed position to facilitate the protector conforming to a body contour. In these embodiments the closure is configured to expand along a hinge line to maintain or increase the volume covered by the closure when moving from the flat position to the flexed position.

The lid panel may include an expansion flange or slip flange which allows the lid panel to expand during movement from the flat position to the flexed position. In one embodiment the lid panel is in two pieces, one disposed on either side of a hinge, and a tongue extends from one lid panel piece and is received by the other lid panel piece. The arrangement of these embodiments in operation provides an advantage that the cannulation site is protected from access by the expansion flange when the lid adopts the flexed position to conform to a contoured body site. Advantageously the volume is maintained or increased when the closure is in the flexed position.

This is a function of the geometry of the shield arrangement, whereby the hinge line between the two end pivots is set down at the level of the skirt, while the lid panel is not on the hinge line, but spaced therefrom. Therefore, without the expansion flange, and the lid panel elements sliding over one another, there would be gaps in which fingers could enter the cannulation zone, and potentially be stuck when hinging the cannula protector from a conformal position to a flat position.

In some embodiments the closure flex axis and the chassis hinge axis are collocated. In these embodiments the closure cooperates with a hinge on the chassis when the closure moves to the closed position on the chassis. In some embodiments, the closure flexing to the flexed position is facilitated by a guide adjacent the chassis hinge, the closure following the guide.

In an embodiment the closure includes a latch which is configured to latch over a catch disposed on a side wall opposite the ones on which the lid pivots are disposed. The latch is arranged to be biased to a latched position. The latch includes a sliding actuator which is configured to slide to a release position against a biasing force.

Advantageously, the present technology is useful in normal patient scenarios, but flexible enough to extend to many and varying patient scenarios such as for example: demented & delirious patients; those with an altered level of consciousness such as from head injuries; surgical & ICU patients; children, especially toddlers; those with “precious” lines such as obese patients; diabetic patients; those with fistulas undergoing haemodialysis; those receiving chemotherapy or undergoing diagnostic techniques where IV contrast dye is used; those with “long lines” (devices that are inserted into the arm, but where the tip of the device ends near the heart); intravenous drug users; patients who have few accessible veins.

Advantageously, embodiments of the present technology are adaptable to substantially any site the clinician chooses. The device is robust enough to stay with a patient throughout their hospital stay, moving across from one IV site to another as needed.

Advantageously, embodiments are comfortable, easy to apply, semi-tamper-proof, while allowing easy access for clinicians.

### BRIEF DESCRIPTION OF THE DRAWINGS

In order to enable a clearer understanding, an embodiment of the technology will now be further explained and illustrated by reference to the accompanying drawings, in which:

FIG. 1 is an isometric view from the front and one end of an embodiment of the present technology, being disposed in a flat position and having a closure in a closed position;

FIG. 2 is an isometric view of FIG. 1, but with the closure in an open or access position;

FIG. 3 is an end elevation view of another embodiment of the device shown in FIG. 2 (closure open);

FIG. 4 is an end elevation view of the device shown in FIG. 3 (closure closed and in a pivoted or conformal position);

FIG. 5 is an isometric view of the device as shown in FIG. 3, with the device in a flexed position to accommodate a convex body cannulation site, the closure in the closed position;

FIG. 6 is an isometric view of the device shown in FIG. 5 but from the rear;

FIG. 7 is an isometric view of the device shown in FIG. 6 but with the device in the flat position;

FIG. 8 is a plan view of the device shown in FIGS. 1 and 2;

FIG. 9 is a plan view of the device shown in FIG. 3 (flat position, closure open);

FIG. 10 is an end elevation view of the device shown in FIG. 9 (flat position and closure open);

FIG. 11 is an underside plan view of the device shown in FIGS. 1 and 2;

FIG. 12 is an end elevation view as shown in FIGS. 1 and 2 but with the device in the flat position and closure in the closed position;

FIG. 13 is a plan view of the device shown in FIG. 3, installed with a cannula, closure in the open position;

FIG. 14 is an isometric view of the device shown in FIG. 13 from the front and installed with the cannula as in FIG. 13, closure in the option position;

FIG. 15 is a rear elevation view of the embodiment of device shown in FIG. 1; and

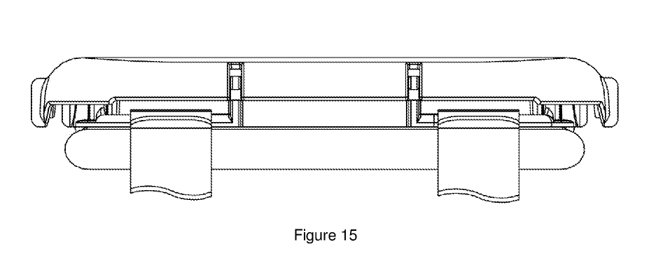

FIG. 16 is a front elevation view of the embodiment of device shown in FIG. 1.

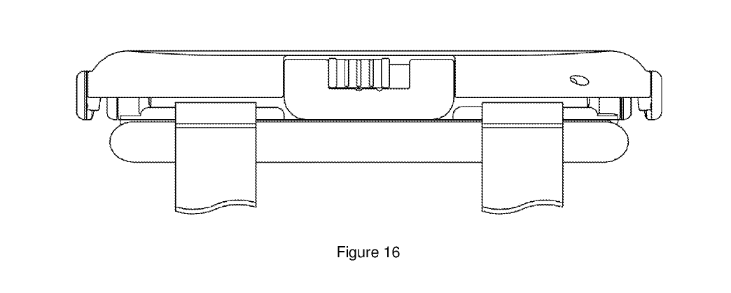

### DETAILED DESCRIPTION OF AN EMBODIMENT

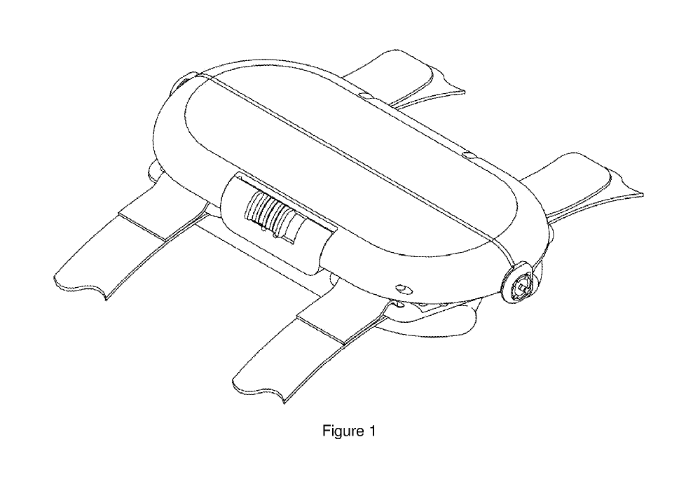

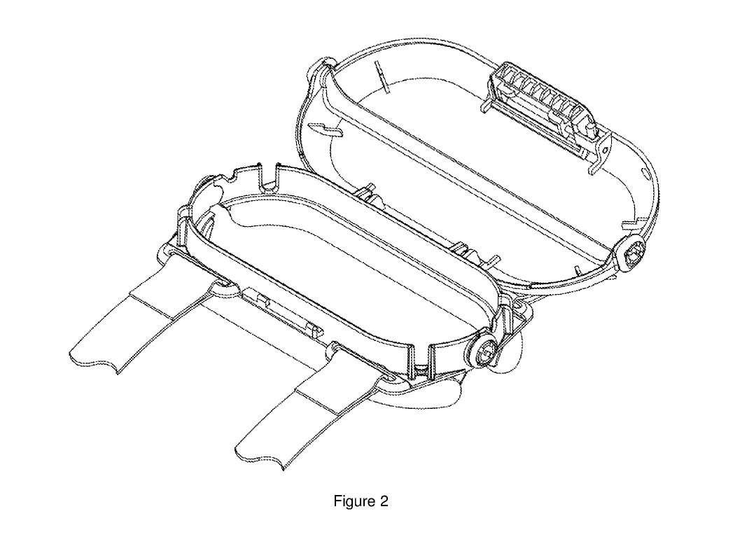

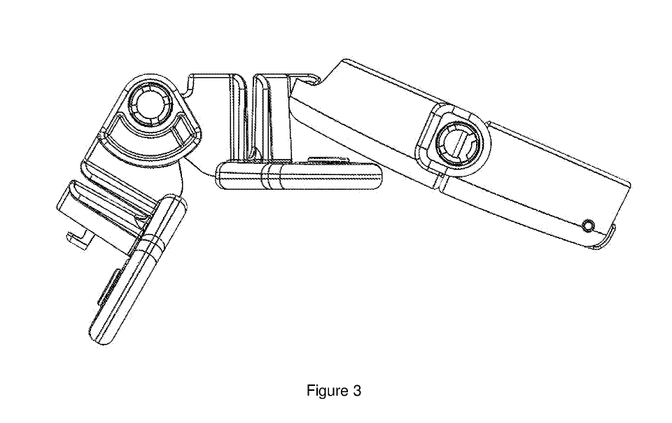

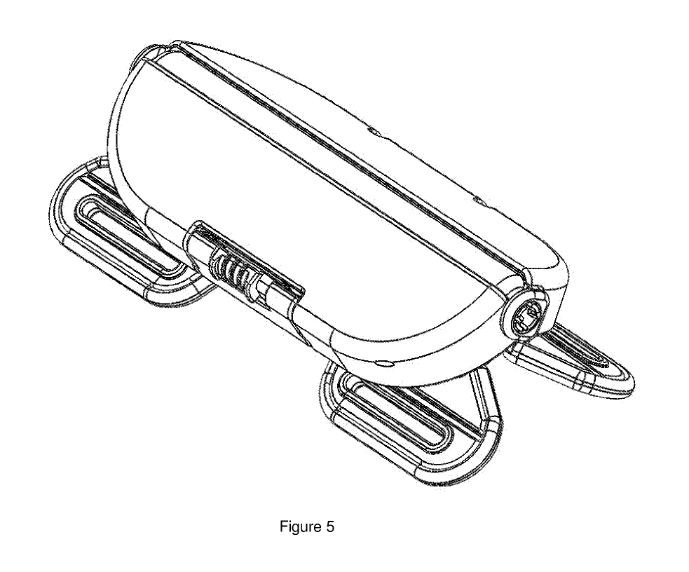

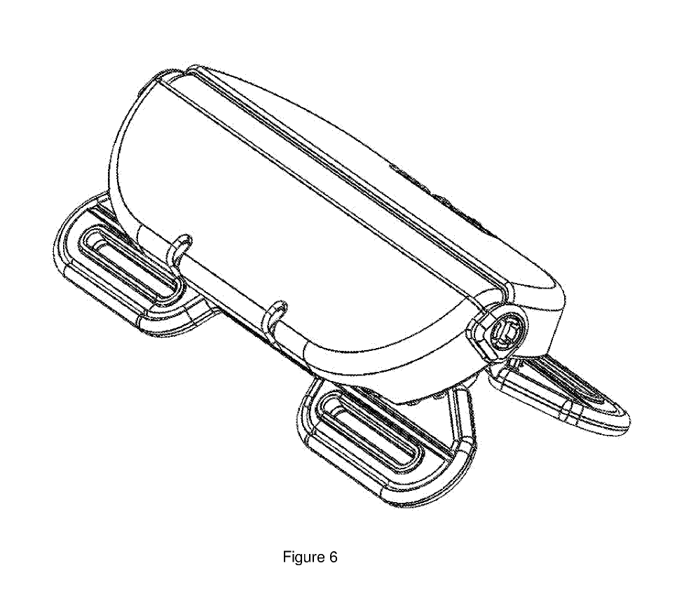

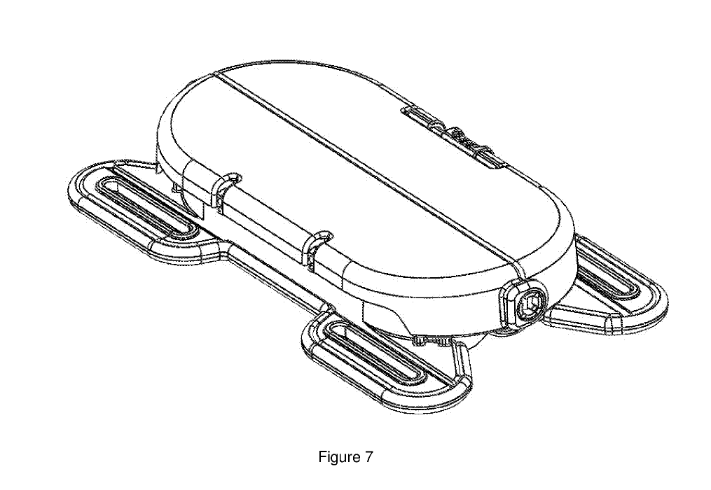

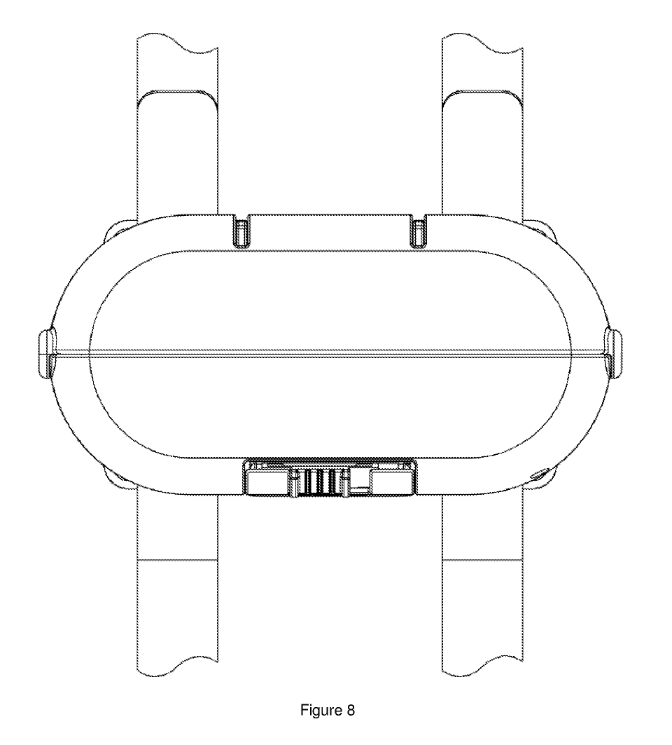

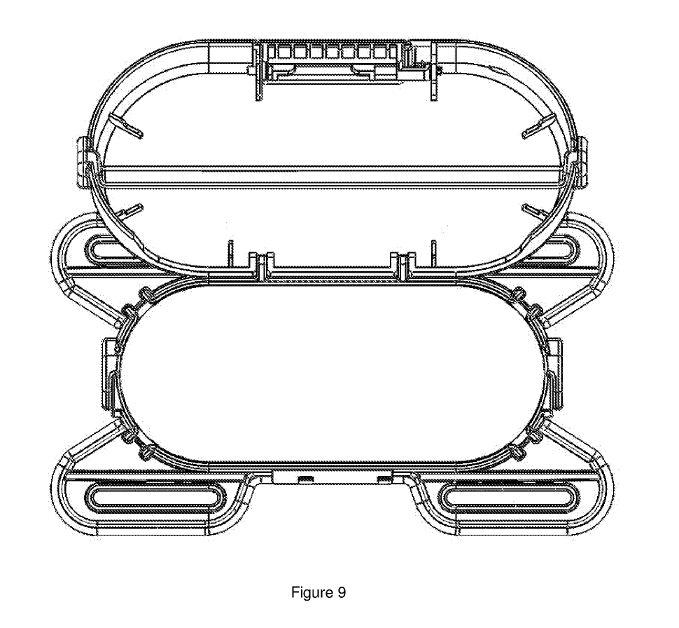

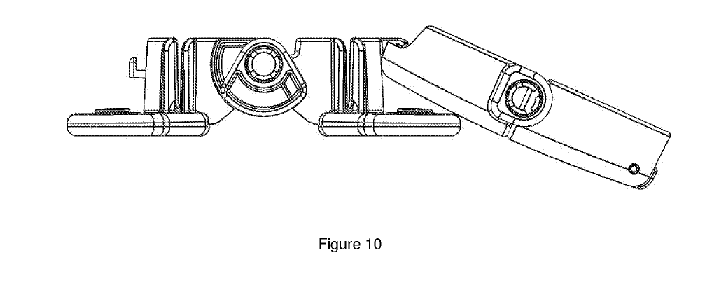

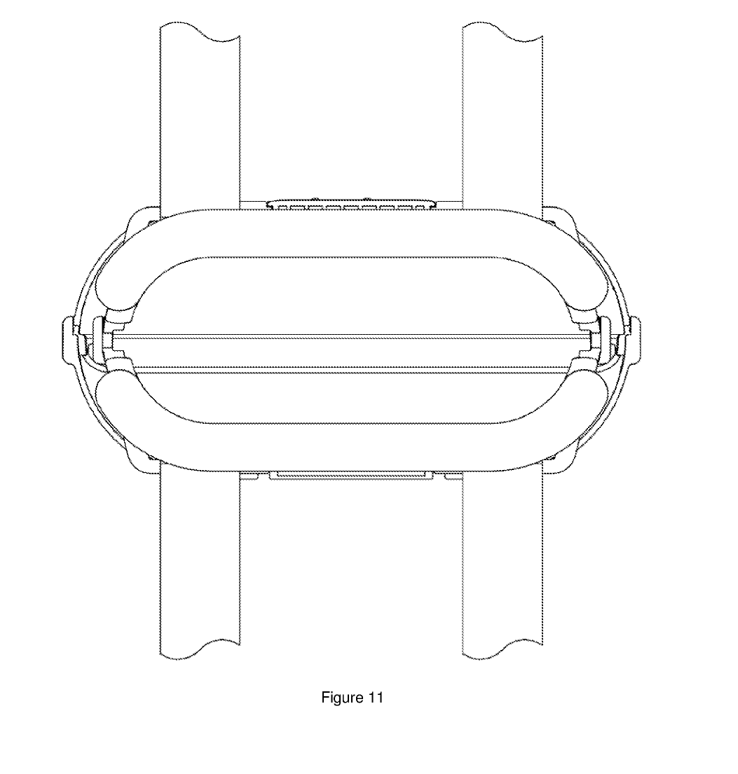

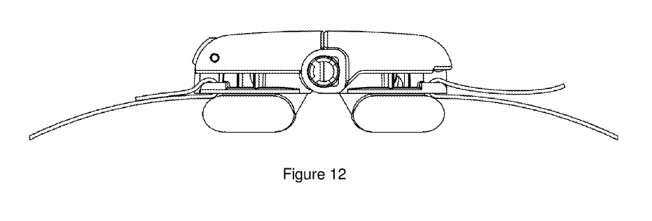

Referring to FIGS. 1 to 14 there is shown a protector generally indicated at 10 for protection of a cannulation site 5 on a surface of a body, where a cannula 7 is inserted into a body vessel near the surface of the body. The protector 10 includes a shield 12 for inhibiting access to the cannula and/or cannulation site and a body mount 14 operatively connected to the shield 12, the body mount 14 configured to provide one or more relief zones 20 so as to relieve the mounting force of the protector 10 on one or more portions of the body when the shield is mounted on the body.

Generally speaking, the body mount 14 is configured as a plurality of body engaging support elements or support portions or feet 16 that are disposed around a periphery of the shield 12, and at least two of the body engaging support elements or support portions or feet 16 are spaced apart from one another to provide at least one of the relief zones 20 therebetween.

In the embodiment shown, the plurality of support elements 16 is arranged on or adjacent a periphery of the shield 12 such that, when the protector 10 is in an installed position on the cannulation site, one or more support elements engage the body on one side of the body vessel, and one or more support elements engage the body on the other side of the vessel. In this manner, pressure on the vessel is relieved, so as to facilitate patency of the vessel.

### Body Mount

### Supports 15

The arrangement shown in the drawings is such that the body mount 14 includes a plurality of supports 15 for supporting the shield 12 in a selected position on the cannulation site on the body. The supports 15 include feet 16 for distributing loads of the shield 12 on the body. The feet 16 are spaced apart from one another to provide stability to the shield 12. Where there are provided shield vertices 31, the feet are spaced proximal opposed vertices of the shield for facilitating greater stability. The feet 16 include foot plates 17 for further distributing loads of the shield 12 on the body over a greater surface area of the body.

The feet 16 include a fastener mount 18 for mounting a fastener 11. The fastener mount 18 is a webbing strap receiver for receiving a webbing strap 9. The webbing receiver in the embodiment shown, an aperture 19 but it could, with similar efficacy, be some other form of guide including posts or channels, or other fastener mount that could be useful for holding the feet 16 against the cannulation site on the body. The fastener mount could be in some embodiments, an adhesive receiving area, or a tape hold-down area, or elastic band area, or other clamp for holding to a body to protect the cannulation site.

The feet 16 include a pliable relief material 17 for relieving and distributing stresses on particular portions of the feet on the body and so as to expand to fill any gaps which may be opened by undulations in the body adjacent the cannulation site. This gap-filling function further inhibits access to the cannulation site since the patient is inhibited from reaching between their own skin and the shield 12. The pliable material may be integrated with or applied to the surface of the feet. The pliable material may be EVA foam or other suitable foam or rubber, silicone, and spread evenly on the surface of the feet or applied in pads. There equally may be other pads or bosses or other extension pieces or fingers which when spread in discrete places on the feet, may distribute loads on the body. The bosses or fingers may be formed from a deformable material, or formed sufficiently finely so as to be flexible (plastic fingers, even metallic springs if suitably finely formed, and, say, leaved).

The feet 16 themselves may also conform to some contour of the body adjacent the cannulation site. Thus, the feet are articulated, or hinged in some way such as by having an integrated or living hinge 7. The feet have an outer portion 22, in which the fastener mount 18 is disposed, and which is distal the shield 12, and which is movable between a flat position (shown in all the drawings) and a flexed position (not shown) which in this embodiments may be up to 90° to improve the distribution of the foot load and to inhibit access to the cannulation site 5 when required by the contours of the body adjacent the cannulation site. For example, on a small human forearm, and the cannulation site is on a blade of the forearm, all three hinges (feet hinge 17, chassis hinge 50, cover hinge 78) might move in an aligned way at least some way to their fully flexed positions to provide increased conformance and inhibit access to the cannulation site, without pressing too hard on the vessel itself.

### Chassis 40

The support includes a chassis 40 to provide spatial placement of other elements, including the shield 12.

The chassis 40 is configured to space the feet 16 apart from one another to facilitate stability of the shield 12. Thus, the chassis 40 includes chassis walls 42 to inhibit access to the cannulation site. The chassis walls 42 include spaced-apart side chassis walls 44 and spaced-apart end chassis walls 46, operatively connected together. An opening 45 is provided, disposed at a top of the walls such that the IVD line, or the cannulation site generally, can in use be accessed therethrough.

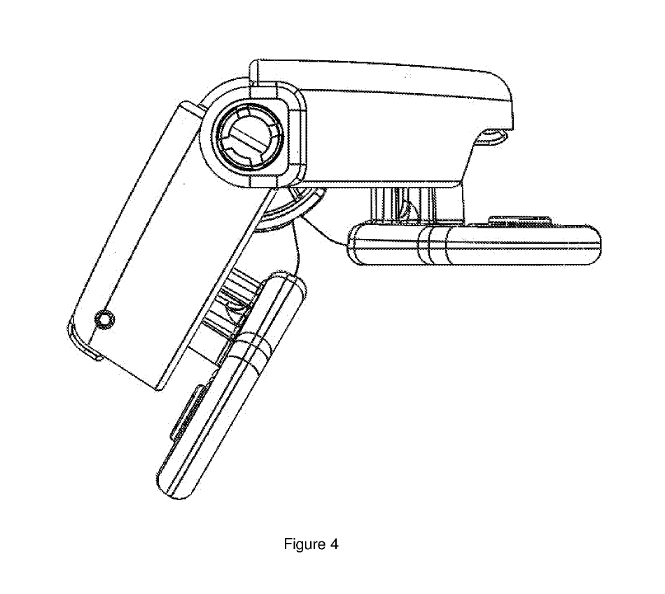

The chassis 40 is configured to facilitate the shield 12 conforming to the body. The chassis 40 is articulated to facilitate the chassis moving between a straight position (FIG. 1, for example) and a flexed position (FIG. 4, for example) in which it may conform to some convex surface such as a forearm. The chassis 40 may be flexible in some way such that some first chassis elements 47 can be positioned in some selected flexed angular position relative to other chassis elements 49, or, some of the first chassis elements 47 can be positioned in some flexed angular position relative to the other chassis elements 49. The chassis 40 includes a chassis hinge 50 to facilitate articulation.

The chassis walls 42 include one or more inlets 55 for facilitating inlet of an IV line 60. There are four inlets 55 provided, disposed in opposed vertices of the chassis 40. The inlets 55 include buttresses 56 to strengthen the chassis walls 42 and to reduce snags for the IV line 60.

### Chassis-and-Support Interaction: Relief Zone 20

One foot 16 is mounted at either end of a spaced-apart side chassis wall 44 to facilitate stability of mounting. There is provided a foot extension pad 13 between the two feet 16, which extends along an underside of each spaced-apart side wall to further inhibit access to the cannulation site 5. The foot extension pad 13 includes soft EVA foam as described above or a hollow air cushion.

The feet 16 extend beyond the spaced-apart side walls 44 so as to inhibit access to the cannulation site 5.

The spaced-apart end chassis walls 46 are arranged such that there is a cutout in that a base edge extends upwards from the feet 16 to provide one form of relief zone thereunder. This assists with pinch free operation when the hinge is in the conformal position or flexed position. The relief zone 20 is where there is an air gap disposed in use between the body and the body mount 14. The relief zone 20 facilitates patency of a vein or artery or other fluid channel within the body, near the surface of the body, adjacent the protector 10. This is useful to facilitate IVD fluid delivery to other parts of the body.

The chassis hinge 50 is disposed in each of the spaced-apart end walls 46 to form a chassis hinge line 51 extending between the end chassis walls 46 and parallel to the side chassis walls 44. The chassis hinge 50 is arranged in the spaced-apart end chassis walls 46 such that the chassis hinge 50 is vertically spaced from the feet to inhibit pinching between feet when the chassis moves to a flexed position.

The feet 16 are configured to articulate along a foot hinge line 7 which is parallel to the chassis hinge line 51 so as to further facilitate conforming of the protector 10 to the body.

The end chassis walls 46 include a guide 47 for the shield 12 so that the shield 12 may be guided in its articulation.

Since the end chassis walls 46 include the relief zone, the end chassis walls 46 further include a flange 49 disposed thereon to cover at least the higher portions of the relief zone to inhibit access to the cannulation site therethrough. The flange 49 is in some embodiments integrated with the guide 47.

### Shield 12

The shield 12 includes a closure 70 which is configured to close the chassis opening 45, by adopting a closed position (FIG. 1, for example), to inhibit access to the cannulation site or the IVD itself. The closure 70 includes a lid panel 72. The lid panel 72 includes a downwardly depending skirt 74 having opposed walls configured to cooperate with the opposed walls of the chassis by being spaced outwardly therefrom and extending parallel thereto when in the closed position. The skirt 74 further inhibits access to the cannulation site 5 by covering the plurality of inlets 55, increasing its strength, and also by inhibiting a patient prising off the lid panel from the chassis 40.

The closure 70 is movable between the closed position and an access position. The closure may be movable between the closed position and the access position by being detachable, or slidable, or hinged. The lid panel 72 is formed in two lid panel elements 71 and 73, wherein lid panel 71 pivotally attaches to the chassis on joint line 76 to form a hinged closure so the closure can be operated with one hand.

The closure 70 is pivotally attached to chassis 40 at joint line 76 to one side wall 46 so as to facilitate ease of access to the cannulation site 5. The arrangement is such that the closure hinges on closure hinge line 76 parallel to the chassis hinge line 51 for simplicity of operation and for strength of hinge—since the hinges can be spaced further apart along the side wall than the end wall, the closure is more secure than if it were hinged on that end wall.

The closure may be translucent, transparent, or partially transparent, or transparent on one lid panel and translucent on the other, or transparent on both.

### Expanding Closure 70

The closure 70 is also configured to move between a flat position and a flexed position to facilitate the protector conforming to a body contour. The closure 70 is thus configured to expand along one surface to maintain or increase the volume covered by the closure when moving from the flat position to the flexed position.

The lid panel 72 articulates along joint 78, which in the embodiments shown includes an expansion flange 77 or slip flange which allows the lid panel 72 to expand during movement from the flat position to the flexed position. In one embodiment the lid panel is in two pieces, one disposed on either side of a hinge 78, and a tongue extends from one lid panel piece (in this case 73) and is received by the other lid panel piece 71. The arrangement of these embodiments in operation provides an advantage that the cannulation site is protected from access by the expansion flange when the lid adopts the flexed position to conform to a contoured body site. Advantageously the volume is maintained or increased when the closure is in the flexed position. There may be two, three, four, five, six, seven, or any suitable number of pieces of the lid panel 72 or lid panel elements.

In some embodiments the closure flex axis and the chassis hinge axis are collocated. In these embodiments the closure cooperates with a hinge on the chassis when the closure moves to the closed position on the chassis. In some embodiments, the closure flexing to the flexed position is facilitated by a guide adjacent the chassis hinge, the closure following the guide.

The closure 70 includes a latch 90 which is configured to latch over a catch 92 disposed on the other side wall. The latch slides along side wall 44 which adds an element of tamper proofing and is arranged to be biased to a latched position toward one end 46. The latch 90 includes a sliding actuator 91 which is configured to slide to a release position against a biasing force provided by a spring (not shown).

The apertures 55 are sized such that in operation the line connector 8 which connects the cannula line 3 to the main fluid introduction line 60 are inhibited from being pulled through.

### Application to Body Part

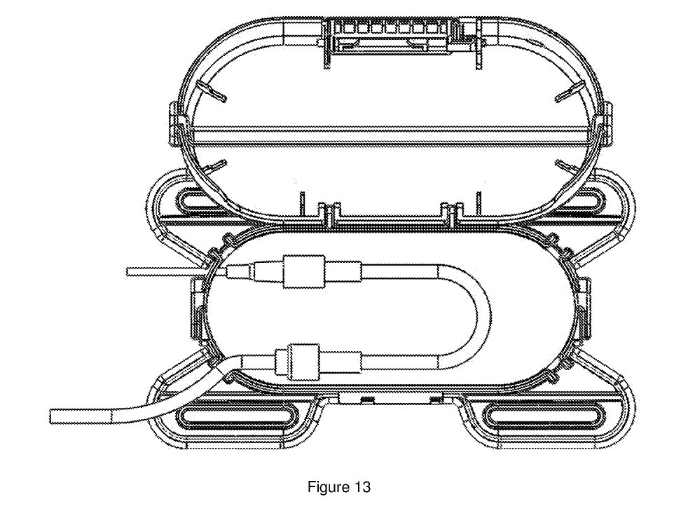

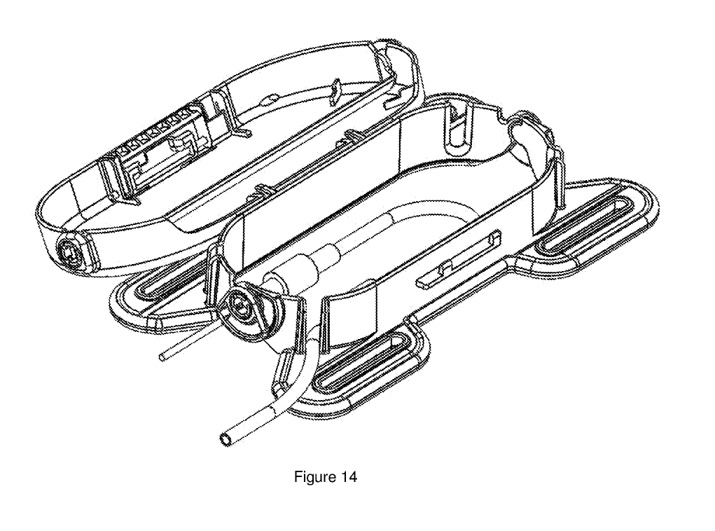

In operation, as shown in at least FIGS. 13 and 14, cannula 6 is applied to a limb of a patient at a cannulation site so that the vein or artery is accessible to the cannula line 3. The connector 8 has a check valve to stop release of blood from the vein or artery therefrom and is larger in diameter than the inlets 55 so it catches in the inlets 55 when the line 60 is pulled, inhibiting cannula release from the limb, so long as there is some play (a little loop in the line 3 to follow the contour of the chassis end walls 46). This is one advantage of having a curved end wall 46—it can function as a rest/guide for the cannula line 3.

The lid of the protector 10 is opened by opening the latch 90 and swinging the closure 70 about pivots on hinge line 76 and then the protector 10 is applied to the patient around the cannulation site 5 by positioning the body mounts 14 around the cannula 6, generally, where possible, ensuring the protector 10 is oriented such that relief zones 20 are placed above the cannula 6 and/or any veins or arteries or other lines to facilitate their patency during introduction of intravenous treatment through the cannula 6.

The protector 10 may be positioned to match the contour of the limb by flexing about the axes 7/50. The lid is closed, and depending on the flex of the axis 50, the lid will flex about axis 78, extending the surface of the lid panel via the slip joint 77.

The straps 9 are tightened and the protector 10 is secure.

### Advantages of One or More Embodiments

Embodiments of the present invention provide, among other things, protection from mechanical force directly on the cannula 6 (shield 12); protection from indirect force pulling on the line 60; ease of access to cannulation site 5 (closure 70 hinged at 76); visibility of cannulation site 5 (transparent closure 70); comfort for patients (padded feet); repeatable safety relatively independent of skill level; an element of tamper proofing (sliding latch 90).

In this specification, where a document, act or item of knowledge is referred to or discussed, this reference or discussion is not an admission that the document, act or item of knowledge or any combination thereof was at the priority date:
(a) part of common general knowledge; or
(b) known to be relevant to an attempt to solve any problem with which this specification is concerned.

The word ‘comprising’ and forms of the word ‘comprising’ as used in this description do not limit the invention claimed to exclude any variants or additions.

Modifications and improvements to the invention will be readily apparent to those skilled in the art. Such modifications and improvements are intended to be within the scope of this invention.

The claims defining the invention are as follows:

1. A cannula protector for protection of a cannulation site on a body, the protector including:
a shield for inhibiting access to the cannulation site; and
a body mount operatively connected to the shield to mount the shield on the body, the body mount configured to provide one or more relief zones so as to relieve the mounting force of the protector when mounted on the body.

2. The cannula protector in accordance with claim 1 wherein the body mount is a plurality of body engaging mounting elements which are disposed adjacent or around a periphery of the shield, and at least two of the body engaging support elements or portions are spaced apart from one another to provide at least one of the relief zones therebetween.

3. The cannula protector in accordance with claim 1 wherein the shield includes a lid panel in the form of a plurality of lid elements overlapping one another and configured to move in a sliding relationship relative to one another to maintain a cover over the cannulation site when the shield is moved from a flat position to a conformal position.

4. The cannula protector in accordance with claim 1 or 2 wherein the body mount and the shield are configured to conform to a contour of the body.

5. The cannula protector in accordance with any one of the previous claims wherein the body mounts include feet for distributing loads of the shield on the body.

6. The cannula protector in accordance with claim 5 wherein the feet are elongate in that they include plates or plate-shaped foot elements for more widely distributing the loads of the shield on the body over a greater surface area of the body.

7. The cannula protector in accordance with any one of the previous claims wherein the body mount includes a fastener mount for mounting a fastener.

8. The cannula protector in accordance with claim 7 wherein the fastener mount is a webbing receiver for receiving a webbing strap.

9. The cannula protector in accordance with any one of claims 5 to 7 wherein the feet include a pliable, spongy or soft material for further distributing stresses from particular portions of the feet on the body and so as to expand to fill any gaps which may be opened by undulations in the body adjacent the cannulation site.

10. The cannula protector in accordance with claim 9 wherein the pliable material is a pillow or air cushion.

11. The cannula protector in accordance with any one of claims 5 to 10 wherein the feet are articulated.

12. The cannula protector in accordance with any one of the previous claims wherein a liner on the body mounts is provided to inhibit and/or absorb sweat developing under the pillow elements while in use.

13. The cannula protector in accordance with any one of the previous claims wherein the body mount includes a chassis or frame to facilitate the spatial placement of other elements, including the shield.

14. The cannula protector in accordance with claim 13 wherein the chassis is a peripheral frame arrangement having one or more frame members.

15. The cannula protector in accordance with claim 13 or 14 wherein the chassis includes chassis walls to inhibit access to the cannulation site.

16. The cannula protector in accordance with claim 15 wherein the chassis walls include end walls which are curved to guide installation of a cannula line.

17. The cannula protector in accordance with any one of claims 13 to 16 wherein the chassis is articulated to facilitate the chassis moving between a straight position and a flexed position in which the chassis may conform to some convex surface such as a forearm.

18. The cannula protector in accordance with any one of claims 15 to 17 wherein the chassis walls include one or more inlets for facilitating inlet of an IV line to the cannulation zone.

19. The cannula protector in accordance with claim 19 wherein the inlets include buttresses to strengthen the chassis walls and to reduce snags for the IV line.

20. The cannula protector in accordance with any one of claims 16 to 19 wherein the chassis end walls are arranged such that there is a cut away in the chassis end walls such that the base edge extends upwards from the feet to provide pinch-free operation and a relief zone adjacent the chassis hinge line.

21. The cannula protector in accordance with any one of the previous claims wherein a relief zone is provided in the form of a spongy area under one or more body mounts.

22. The cannula protector in accordance with any one of the previous claims wherein the shield includes an articulated lid panel which includes a skirt depending downwardly therefrom having opposed skirt walls configured to cooperate with the opposed chassis walls by being spaced outwardly therefrom and extending parallel thereto when in the closed position.

23. The cannula protector in accordance with any one of the previous claims wherein the shield is pivotally attached to one chassis side wall to form a hinged closure.

24. The cannula protector in accordance with any one of the previous claims wherein the shield includes a plurality of pivoting lid panel elements, each one overlapping and sliding over the adjacent lid panel element.

25. The cannula protector in accordance with any one of claims 13 to 24 wherein the closure is configured to move with the chassis, the closure being configured to expand along a lid panel surface.

26. The cannula protector in accordance with claim 25 wherein the lid panel includes an expansion flange which allows the lid panel to expand during movement from the flat position to the flexed position.

27. A protector for protection of a cannulation site on a body, the protector including:
a shield for inhibiting access to the cannulation site; and
a body mount operatively connected to the shield, the body mount including a plurality of body engaging feet;
wherein the body engaging feet are configured to conform to a contour of the body to inhibit access to the cannulation site.
